(19) United States
(12) Patent Application Publication
(10) Pub. No.: US 2014/0276791 A1
(43) Pub. Date: Sep. 18, 2014

(54) Treatment Device With Electrode Contact Surface Configured for Enhancing Uniformity of Electrical Energy Distribution and Associated Devices and Methods

(71) Applicant: Medtronic Ardian Luxembourg S.a.r.l., Luxembourg (LU)

(72) Inventors: Vincent W. Ku, Palo Alto, CA (US); Joan A. Simon, Sunnyvale, CA (US); Andrew E. Wu, Los Altos Hills, CA (US); Mingfei Chen, Santa Rosa, CA (US); Nancy P. Nguyen, San Jose, CA (US)

(73) Assignee: Medtronic Ardian Luxembourg S.a.r.l., Luxembourg (LU)

(21) Appl. No.: 14/208,590

(22) Filed: Mar. 13, 2014

Related U.S. Application Data
(60) Provisional application No. 61/800,535, filed on Mar. 15, 2013.

(51) Int. Cl. A61B 18/14

(52) U.S. Cl. CPC A61B 18/1492; A61B 2018/00434
USPC 606/41

(57) ABSTRACT

Treatment devices with electrode contact surfaces configured for enhancing uniformity of electrical energy distribution are provided. In one embodiment, a treatment device includes a tubular electrode having a wall, a contact surface defined by the wall, and cut shapes at least partially extending through the wall. The tubular electrode is configured to transmit electrical energy to a treatment site within a body lumen via the contact surface, and the individual cut shapes are configured to draw a portion of the electrical energy toward an interior region of the contact surface. A shaft having a distal end portion operably coupled to the tubular electrode can locate the tubular electrode at the treatment site.

### CROSS-REFERENCE TO RELATED APPLICATION(S)

This application claims the benefit of currently pending U.S. Provisional Patent Application No. 61/800,535, filed Mar. 15, 2013, the disclosure of which is incorporated herein by reference in its entirety.

### TECHNICAL FIELD

The present technology is related to treatment assemblies including electrodes that can be deployed for executing therapeutic treatments (e.g., neuromodulation treatments) within body lumens.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. Excessive activation of the renal SNS in particular has been identified experimentally and in humans as a likely contributor to the complex pathophysiology of hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function are considerably stimulated in disease states characterized by heightened sympathetic tone. For example, reduced renal blood flow and glomerular filtration rate as a result of renal sympathetic efferent stimulation is likely a cornerstone of the loss of renal function in cardio-renal syndrome, (i.e., renal dysfunction as a progressive complication of chronic heart failure). Pharmacologic strategies to thwart the consequences of renal sympathetic stimulation include centrally-acting sympatholytic drugs, beta blockers (e.g., to reduce renin release), angiotensin-converting enzyme inhibitors and receptor blockers (e.g., to block the action of angiotensin II and aldosterone activation consequent to renin release), and diuretics (e.g., to counter the renal sympathetic mediated sodium and water retention). These pharmacologic strategies, however, have significant limitations including limited efficacy, compliance issues, side effects, and others.

### BRIEF DESCRIPTION OF THE DRAWINGS

Many aspects of the present technology can be better understood with reference to the following drawings. The relative dimensions in the drawings may be to scale with respect to some embodiments. With respect to other embodiments, the drawings may not be to scale. For ease of reference, throughout this disclosure identical reference numbers may be used to identify identical or at least generally similar or analogous components or features.

FIG. 1 is a partial, isometric view of a treatment device having a tubular electrode with a contact surface configured in accordance with an embodiment the present technology.

FIG. 2A is partial, top-plan view of a portion of the tubular electrode of FIG. 1 showing cut shapes within the contact surface.

FIG. 2B is a partial, top-plan view of the portion of the tubular electrode shown in FIG. 2A during energy delivery showing field points corresponding to an electrical field in the vicinity of the cut shapes.

FIGS. 3A-3C are partial, top-plan views showing various cut shapes configured in accordance with embodiments of the present technology.

FIG. 4 is a partial, top-plan view showing differently sized cut shapes configured in accordance with an embodiment of the present technology.

FIG. 5 is a partial, top-plan view showing differently spaced cut shapes configured in accordance with an embodiment of the present technology.

FIG. 6 is a partial, top-plan view showing inner and outer dielectric material portions configured to define contact surface portions of a tubular electrode configured in accordance with an embodiment of the present technology.

FIG. 7 is a partial, top-plan view showing inner and outer dielectric material portions configured to define a contact surface of a tubular electrode configured in accordance with an embodiment of the present technology.

FIGS. 8A and 8B are partially schematic, cross-sectional anatomical views illustrating operation of the treatment device of FIG. 1 in accordance with an embodiment of the present technology.

FIGS. 8C and 8D are partially schematic, cross-sectional anatomical views illustrating lesions profiles that can be achieved with various configurations of cut shapes and dielectric materials in accordance with an embodiment of the present technology.

FIG. 9A is a perspective view of a catheter including a control member and the treatment device of FIG. 1 configured in accordance with an embodiment of the present technology.

FIG. 9B is a perspective view showing an alternative configuration of the control member of FIG. 9A configured in accordance with another embodiment of the present technology.

### DETAILED DESCRIPTION

The following disclosure describes intravascular treatment devices having an electrode contact surface configured for at least generally uniform delivery of electrical energy and associated devices, systems, and methods. Specific details of several embodiments of the present technology are described herein with reference to FIGS. 1-9B. Although several of the embodiments are described in the context of intravascular neuormodulation of renal tissue, other applications and other embodiments in addition to those described herein are within the scope of the present technology. For example, some embodiments may be useful for treatment of cardiac conditions (e.g., atrial fibrillation) or cosmetic conditions (e.g., varicose veins). Additionally, some embodiments may be useful in therapies applied outside of the vasculature, such as within the digestive tract (e.g., for Barret's esophagus). Additionally, some embodiments of the present technology can have different configurations, components, or procedures than those shown or described herein. Moreover, a person of ordinary skill in the art will understand that some embodiments can have components and/or procedures in addition to those shown or described herein and that these and other embodiments can be without several of the components and/or procedures shown or described herein without deviating from the scope of the present technology.

In some instances, conventional catheter electrodes can over-deliver or under-deliver electrical energy. This can occur, for example, due to an “edge-effect” at a contact surface of an electrode. For example, a localized region of relatively high electrical field strength (e.g., current density), or “hot spot,” may develop at the edge of a contact surface and a localized region of relatively low electrical field strength, or “cold spot,” may develop toward an interior of the contact surface. Hot spots can damage non-target tissue, while cold spots can undertreat target tissue. To compensate for hot spots, one conventional approach is to increase electrical resistance at or near edges of a contact surface. In effect, this can create a larger resistive path that steers electrical current (and the attendant field) towards less resistive interior portions of the contact surface. This technique, however, typically increases resistive heating, which can increase the probability of damaging non-target tissue. To correct for cold spots, another conventional approach is to deliver more power to a contact surface. Although this can increase the electrical field strength at the cold spots, it also typically further increases the electrical field strength at the hot spots, which likewise can increase the probability of damaging non-target tissue.

At least some embodiments of the present technology provide new ways to compensate for hot spots and/or cold spots at a contact surface. In some embodiments, a tubular electrode includes slots configured to bias expansion of the electrode into a helical expanded form in which a contact surface of the electrode operably engages an inner wall of a body lumen. The slots can have end portions within the contact surface that are shaped to enhance the uniformity of an electrical field over the contact surface during energy delivery toward tissue at the inner wall of the body lumen. In other embodiments, a tubular electrode can include a dielectric material selectively located over portions of a contact surface of the electrode. For example, the dielectric material can be selectively positioned over the contact surface so as to electrically insulate portions of the contact surface characterized by relatively high electrical field strength while leaving other portions of the contact surface exposed. Similar to the shaped slot end portions, this can enhance the uniformity of an electrical field over the contact surface during energy delivery toward tissue at the inner wall of the body lumen. In still other embodiments, a tubular electrode can include both shaped slot end portions and selectively coated dielectric material within a contact surface of the electrode. Characteristics (e.g., shape, size, and spacing, among others) of shaped slot end portions within a contact surface and/or regions of dielectric material coating on a contact surface can be selected to enhance control over the profile, depth, and/or other aspects of lesions formed at a treatment site.

FIG. 1 is a partial, isometric view of a treatment device 100 configured in accordance with an embodiment the present technology. The treatment device 100 can include a tubular electrode 102 having a wall 103 with an outer surface 105 and an inner surface 106. The tubular electrode 102 can be formed from a variety of conductive materials, including, for example, conductive polymers, metallic materials, and/or alloyed materials. In one embodiment, for example, the tubular electrode 102 is formed from nitinol. In other embodiments, the tubular electrode 102 may be formed from two or more different suitable materials. A dielectric material 108 can at least partially cover the outer surface 105. In some embodiments, the dielectric material 108 at least partially defines the shape of a contact surface 110 at a distal end portion 112 of the tubular electrode 102. In other embodiments, the dielectric material 108 can be spaced apart from the contact surface 110. As used herein, the term “contact surface” refers to a conductive surface configured to make physical contact with (or near physical contact with) a target area of tissue at a treatment site. For example, the contact surface 110 of the tubular electrode 102 can be a surface portion of the tubular electrode 102 configured to face toward an inner wall of a body lumen when the tubular electrode 102 is in a deployed state at a treatment location within the body lumen. The tubular electrode 102 can be operably connected to a power source (not shown). During a treatment procedure, the tubular electrode 102 can be configured to be energized so as to transmit electrical current into tissue at the treatment location via the contact surface 110.

The dielectric material 108 can be a deposited film, a coating, an adhesive layer, a patterned sheet, or have another suitable form. Suitable compositions for the dielectric material 108 include, for example, polymers (e.g., epoxies and polyolefin, among others). In some embodiments, the dielectric material 108 at least partially covers the inner surface 106 of the tubular electrode 102. In addition to being electrically insulative, the dielectric material 108 can be thermally conductive. Polyolefin, for example, can be relatively electrically resistive and relatively thermally conductive. The combination of relatively high electrical resistance and relatively high thermal conductivity can be useful, for example, to facilitate heat transfer away from the tubular electrode 102 (e.g., toward blood flowing past the tubular electrode 102) during a treatment procedure. In some cases, facilitating heat transfer away from the tubular electrode 102 during a treatment procedure can reduce resistive heating of at least a portion of the tubular electrode 102 during a treatment procedure. Such resistive heating can increase the probability of damaging non-target tissue in the vicinity of the tubular electrode 102 and/or cause undesirable conductive heating of target tissue in contact with the tubular electrode 102.

With reference to FIG. 1, the tubular electrode 102 can include slots 115 cut (e.g., laser-cut, etched, sawed, etc.) through the tubular electrode 102 in a direction generally transverse to the X axis (as shown in FIG. 1). The slots 115 can be configured to bias the tubular electrode 102 so that it expands in transverse dimension relative to a longitudinal axis of the tubular electrode 102 in response to an applied force (e.g., in response to tension on an elongated control member (not shown in FIG. 1) operably connected to the tubular electrode 102 at a location distal to the slots 115). In some embodiments, the slots 115 are arranged along the tubular electrode 102 to define, distal-to-proximal, an electrode region 116a, a transition region 116b, and a flexible region 116c. The electrode region 116a alone, in combination with the transition region 116b, or in combination with the transition region 116b and the flexible region 116c, can be configured to expand into a helical or other suitable expanded form. In some embodiments, the individual slots 115 have one or two shaped (e.g., curved, rounded, or otherwise non-squared) end portions or cut shapes 113 within the contact surface 110. In other embodiments, the slots 115 may have end portions that are squared or otherwise not shaped and/or outside the contact surface 110. As described in greater detail below, the cut shapes 113 can be configured to enhance the uniformity of electrical field distribution at the contact surface 110 during a treatment procedure.

FIGS. 2A and 2B are partial, top-plan views of a portion of the tubular electrode 102. Referring first to FIG. 2A, the slots 115 can extend through the dielectric material 108 and toward the contact surface 110 to define interconnected tubular electrode sections 220. The individual slots 115 can include outer edge portions 217 (identified individually as first and second outer edge portions 217a and 217b) outside of the contact surface 110 and inner edge portions 219 (identified individually as first and second inner edge portions 219a and 219b) within the contact surface 110. The outer edge portions 217 and the inner edge portions 219 can be portions of edges around the slots 115 that are at least generally perpendicular to the length of the tubular electrode 102. The slots 115 can further include inner transitional edge portions 219c extending between ends of associated pairs of the first and second inner edge portions 219a, 219b. In the illustrated embodiment, the transitional edge portions 219c together with associated pairs of the first and second inner edge portions 219a, 219b define the cut shape 113. In other embodiments, the transitional edge portions 219c can extend to points outside of the contact surface 110. For example, the first and second inner edge portions 219a and 219b can be absent and the transitional edge portions 219c can extend between ends of associated pairs of the first and second outer edge portions 217a, 217b.

The transitional edge portions 219c can be configured to provide gradual (e.g., curved, rounded, or otherwise non-squared) transitions between associated pairs of the first and second inner edge portions 219a and 219b or the first and second outer edge portions 217a and 217b. Generally, it is expected that the transitional edge portions 219c can mitigate acute focusing of electrical field strength in the vicinity of the cut shapes 113. In the illustrated embodiment, the transitional edge portions 219c are rounded. In other embodiments, the transitional edge portions 219c can have other suitable shapes (e.g., one or more of the shapes shown in FIGS. 5A-5C as described below). With reference to FIG. 2B, during energy delivery, an electrical field can extend across the contact surface 110. To illustrate relative intensity, one example of an electrical field 218 is illustrated in FIG. 2B with small field points 222a and medium field points 222b. The medium field points 222b are representative of the electrical field 218 adjacent to the cut shapes 113 and the inner edge portions 219, and the small field points 222a are representative of decreasing intensity of the electrical field 218 away from the cut shapes 113. During operation, electrical current may tend to be drawn towards the cut shapes 113 due to an edge effect associated with the cut shapes 113. It is expected that because the cut shapes 113 extend inwardly towards an interior of the contact surface 110, the electrical field 218 is likely to be more evenly distributed over the contact surface 110 than if the cut shapes 113 were not present. For example, if the slots 115 terminated in squared ends at a periphery of the contact surface 110, it is expected that electrical field strength in the vicinity of the squared ends would be relatively high, thereby causing these areas to act as undesirable hot spots.

It is expected that the electrical field strength and/or density at the cut shapes 113 can be controlled by variously configuring the inner edge portions 219 and the transitional edge portions 219c. In some cases, the effect of the cut shapes 113 may reduce or eliminate the need to increase power, alter resistance, or take other measures to compensate for hot spot, cold spots, or other types of undesirable non-uniformity in an electrical field extending over the contact surface 110. As described in greater detail below, the cut shapes 113 can also be utilized to form a desired profile of a lesion at a treatment site. Similarly, the dielectric material 108 can be configured to form a desired profile of a lesion at a treatment site alone or in combination with the cut shapes 113.

FIGS. 3A-7 provide various examples for configuring cut shapes, tubular electrode segments, and dielectric materials at contact surfaces in accordance with embodiments of the present technology. With reference to FIG. 3A, a first contact surface 302 can include an undulating cut shape 313a having a first transitional edge portion 319a. With reference to FIG. 3B, a second contact surface 303 can include a half-oval cut shape 313b having a transitional edge portion 319b. With reference to FIG. 3C, a third contact surface 304 can include an expanded half-oval cut shape 313c having a transitional edge portion 319c. The cut shapes 313a-c can be configured to control the distribution of electrical field strength and/or density over the contact surfaces 302, 303, 304, respectively. Relative to the cut shapes 113, the cut shapes 313a-c can have larger transitional edge portions 319a-c. As discussed above, it is expected that the cut shapes 313a-c can be configured to enhance the uniformity of electrical current transmission via the contact surfaces 302, 303, 304, respectively, by drawing more (or less) electrical current toward interior regions of the contact surfaces 302, 303, 304.

FIG. 4 is a partial, top-plan view showing a portion of a tubular electrode 400 configured in accordance with an embodiment of the present technology. The tubular electrode 400 can have a contact surface 401 and slots 402 terminating at cut shapes 413 within the contact surface 401. The cut shapes 413 can include first cut shapes 413a and second cut shapes 413b havening different sizes. For example, the first cut shapes 413a can be larger than the second cut shapes 413b. In some embodiments, the first cut shapes 413a are closer to a distal end portion 405 of the tubular electrode than the second cut shapes 413b. In other embodiments, the second cut shapes 413b can be closer to the distal end portion 405 than the first cut shapes 413a. During energy delivery, an electrical field 418 can extend across the contact surface 401. As illustrated by the distribution of small and medium field points 222a, 222b in FIG. 4, it is expected that the electrical field 418 may have a greater strength toward the first cut shapes 413a and lesser strength toward the second cut shapes 413b.

FIG. 5 is a partial, top-plan view showing a portion of a tubular electrode 500 configured in accordance with another embodiment of the present technology. The tubular electrode 500 can have a contact surface 501 and slots 502 terminating at cut shapes 513 within the contact surface 501 and defining interconnected tubular electrode sections 520. The tubular electrode sections 520 can include first tubular electrode sections 520a and second tubular electrode sections 520b having different lengths along a longitudinal axis of the tubular electrode 500. For example, each of the first tubular electrode sections 520a can have a first length L₁ and each of the second tubular electrode sections 520b can have a second length L₂ that is smaller than the first length L1. In some embodiments, the first and second tubular electrode sections 520 are configured to span different portions of the tubular electrode 500. For example, the first tubular electrode sections 520a can be configured to span near the vicinity of a distal end portion 505 of the tubular electrode 500 and the second series of the tubular electrode sections 520b can span a more proximal portion of the tubular electrode 500. In other embodiments, the first and the second tubular electrode sections 520a, 520b can be interleaved with one another across the longitudinal axis of the tubular electrode 500. During energy delivery, an electrical field 518 can extend across the contact surface 501. As illustrated by the distribution of small and medium field points 222a, 222b in FIG. 5, it is expected that the electrical field 518 may have a greater strength toward the second tubular electrode sections 520b and lesser strength toward the first tubular electrode sections 520a

FIG. 6 is a partial, top-plan view showing a portion of a tubular electrode 600 configured in accordance with an embodiment of the present technology. The tubular electrode 600 can have contact surface portions 601, an outer dielectric material portion 608, inner dielectric material portions 609, and slots 602 terminating at cut shapes 613 within the individual inner dielectric material portions 609 and the individual contact surface portions 601. The contact surface portions 601 can include first, second, and third contact surface portions 601a, 601b, 601c separated from one another by first and second inner dielectric material portions, 608a, 608b. For example, the first inner dielectric material portion 609a can separate the first and second contact surface portions 601a, 601b from one another and the second inner dielectric material portion 609b can separate the second and third contact surface portions 601b, 601c from one another. In some embodiments, the inner dielectric material portions 609 are positioned in the vicinity of every other slot 602. In other embodiment, the inner dielectric material portions 609 are aligned differently, such as outside the vicinity of the slots 602 and/or variably spaced apart from one another along the longitudinal axis of the tubular electrode 600. During energy delivery, an electrical field 618 can extend across the individual contact surface portions 601 and the inner dielectric material portions 609. As illustrated by the distribution of small and medium field points 222a, 222b in FIG. 6, it is expected that electrical field lines may extend through the inner dielectric material portions 609. However, it is expected that the inner dielectric material portions 609 may substantially impede the flow of current towards a treatment site at these portions.

FIG. 7 is a partial, top-plan view showing a portion of a tubular electrode 700 configured in accordance with another embodiment of the present technology. The tubular electrode 700 can have a contact surface 701, an outer dielectric material portion 708, inner dielectric material portions 709, and slots 702 terminating at cut shapes 713 within the individual inner dielectric material portions 709 and the contact surface 701. In some embodiments, the inner dielectric material portions 709 are mechanically isolated from the outer dielectric material 708. For example, a first dielectric material portion 709a at a distal end portion 705 can be mechanically isolated from the outer dielectric material 708. In other embodiments, the inner dielectric material portions 709 may be not isolated from the outer dielectric material 708, but instead can connect with the outer dielectric material 708 (e.g., at one side). For example, a second dielectric material portion 709b can connect with the outer dielectric material 708 in the vicinity of one peripheral portion of the contact surface 701 and a third dielectric material portion 709b can connect with the outer dielectric material 708 in the vicinity of another peripheral portion of the contact surface 701. During energy delivery, an electrical field 718 can extend across the contact surface 701 and the inner dielectric material portions 709. As illustrated by the distribution of small and medium field points 222a, 222b in FIG. 7, it is expected that the electrical field lines may extend through the inner dielectric material portions 709. However, it is expected that the inner dielectric material portions 709 may substantially impede the flow of electrical current towards a treatment site at these portions.

FIGS. 8A-8D are partially schematic, cross-sectional anatomical views illustrating operation of the treatment device 100 in accordance with an embodiment of the present technology. For purposes of clarity, the dielectric material 108, the cut shapes 113, and the slots 115 are not shown. Referring first to FIG. 8A, the treatment device 100 is shown in a low-profile configuration (e.g., a contracted configuration) to facilitate advancement of the treatment device 100 through a guide catheter 832 toward a treatment site at a renal artery 833. In one embodiment, the guide catheter 832 is inserted at a percutaneous access site (e.g., at the femoral artery), and an elongated shaft (not shown in FIG. 8A) advances the treatment device 100 through the guide catheter 832. In the illustrated embodiment, the guide catheter 832 includes a delivery sheath 836 to further facilitate delivery of the treatment device 100.

Referring to FIG. 8B, once the delivery sheath 836 is withdrawn, the tubular electrode 102 of the treatment device 100 can expand in transverse dimension in response to a change in tension on a control member (not shown in FIG. 8B) to position the contact surface 110 toward an arterial wall 838 of the renal artery 833. Once properly placed, electrical energy can be delivered to the arterial wall 838 via the contact surface 110 to form a lesion, such as a lesion suitable for therapeutically effective renal neuromodulation. As used herein, the term “neuromodulation” refers to the application of electrical energy (e.g., AC, DC, pulsed, radiofrequency (RF), etc.) for ablation, necrosis, non-ablative injury, or other suitable electrical or thermal treatment of target nervous tissue or supporting structures thereof.

FIGS. 8C-8D are partially schematic, cross-sectional anatomical views illustrating lesions profiles 805 formed in the arterial wall 838 that can be achieved with tubular electrodes 802 having contact surfaces with various configurations of cut shapes and dielectric materials configured in accordance with an embodiment of the present technology. Referring to FIG. 8C, for example, a first tubular electrode 802a can have a contact surface 803 that defines a first lesion profile 805a. In various embodiments, the contact surface 803 can have shape types (FIGS. 3A-3C), differently-sized cut shapes (FIG. 4), and/or differently spaced cut shapes (FIG. 5) that are configured to uniformly distribute an electrical field to form the first lesion profile 805a. In one embodiment, the contact surface 803 defines a substantially continuous lesion profile (e.g., an unbroken lesion profile extending through the page along the X-axis). For example, the first lesion profile 805a can have a continuous spiral or helical shape that extends through the page along the X-axis. In some embodiments, the electrical energy delivered via the contact surface 803 can define a maximum depth d₁ of the first lesion profile 805a. In this regard, the first depth d₁ can be adjusted (e.g., increased or decreased) by changing aspects (e.g., power, frequency, etc.) of an electrical field extending over the contact surface 803.

Referring to FIG. 8D, a second tubular electrode 802b can have contact surface portions 804 that define a second lesion profile 805b having overlapping lesion segments 806. In some embodiments, the contact surface portions 804 can have various configurations of outer and inner dielectric material portions (FIGS. 6-7) to impede electrical current at outer surface portions 807 of the second tubular electrode 802b. In other embodiments, variously configured cut shapes (FIGS. 3A-5) can localize electrical energy at the contact surface portions 804 of the second tubular electrode 802b. In further embodiments, it is expected that various configurations of inner and outer dielectric material portions can work in combination with cut shapes.

It is expected that some of the aforementioned configurations can be utilized to mitigate a so-called kidney effect in which the depth of a lesion profile expands towards its center (see, e.g., maximum depth d₁ of FIG. 8C). It is expected that by distributing the lesion into segments, the depth of a lesion can be better controlled. For example, it is expected that the individual overlapping lesion segments 806 may have a maximum depth d₂ that is shallower than, e.g., the depth d₁ of the first lesion profile 805a. It is further expected that the individual overlapping lesion segments 806 can create uniformity of this depth. For example, centers of the lesion segments 806 can form with greater depth at centers of the contact surface portions 804 and diminishing depth toward edges of the contact surface portions 804 and extending beyond the edges of the contact surface portions 804 into tissue at the outer surface portions 807. As the lesion segments 806 grow, relatively shallow portions of adjacent lesion segments 806 can merge over the outer surface portions 807 and eventually cause the total lesion depth at the outer surface portions 804 to be comparable to the lesion depth at the centers of the contact surface portions 804. In some embodiments, the electrical energy applied at the contact surface 804 can define the maximum depth d₂ of the first lesion profile 805a. In this regard, changing aspects of the electrical energy can adjust the first depth d₁ (by, e.g., increasing or decreasing the electrical energy).

While not shown in the Figures, it is expected that the edge portions (e.g., the edge portions 219 of FIG. 2) can be adjacent to the arterial wall 838 of the vessel (e.g., at 90° with respect the arterial wall 838). Further, in some of these embodiments depending on the orientation of the deployed tubular electrode and the expansion force of the tubular electrode 102, it is expected that at least a portion of the edge portions can project partially into the arterial wall 838.

In operation, it is expected that various embodiments of the treatment devices disclosed herein can be used to form a lesion or series of lesions (e.g., a helical/spiral lesion) that is fully-circumferential overall, but generally non-circumferential at longitudinal segments of the treatment location. This can facilitate precise and efficient treatment with a low possibility of vessel stenosis. Further, it is expected that some of the aforementioned configurations can be used to cause such a fully-circumferential lesion without the need for repositioning within the target vessel.

FIG. 9A is a broken perspective view of a catheter assembly 950 that includes the treatment device 100. For purposes of clarity, the dielectric material, the cut shapes, and the contact surface are not shown. The catheter assembly 950 includes an elongated shaft 952 (e.g., a stainless steel shaft (only partially shown)), an actuator 953 (e.g., a hand-held, thumb-actuated assembly), and a control member 955 extending through the tubular electrode 102 and the shaft 952 to operably couple the actuator 953 to the treatment device 100 at a distal tip 956. In the illustrated embodiment, the control member 955 includes a wire (e.g., a nitinol wire) that is at least partially covered by a sheath 958 to reduce frictional forces. In other embodiments, other suitable control members can be employed.

At least one energy supply wire 960, also extending through the shaft 952, can electrically connect the tubular electrode 102 to a field generator 962. In one embodiment, the energy supply wire 960 is fixedly attached at the interior of the tubular electrode 102 toward a proximal end 963. For example, the energy supply wire 960 can be soldered, welded, or otherwise coupled at the interior the tubular electrode 102. In another embodiment, the energy supply wire 960 can attach to the exterior of the tubular electrode 102.

Inset FIG. 9B shows an alternative configuration in which the energy supply wire 960 is omitted and the control member 955 is configured to provide electrical power. In this embodiment, the control member 955 can be formed from a conductive material, such as a metallic and/or alloyed material (e.g., nitinol), and the sheath 958 (FIG. 8A) can be formed from an insulative material. An energy supply wire 965 can electrically couple the control member 955 with the field generator 962. In the illustrated embodiment, the energy supply wire 965 can electrically connect with an internal conductive member (not shown) of the actuator 953. For example, the energy supply wire 965 can be configured to pass through a wire spool to connect the energy supply wire 965 with the control member 955. In other embodiments, however, the control member 955 can be connected in a different manner. For example, the energy supply wire 965 can be configured to directly connect with the control member 955. Further, in other embodiments, the control member 955 can be configured to provide other types of electrical connections, such as electrical sensor connections (e.g., for impedance measurements, etc.). For example, in one embodiment the control member 955 may comprise a thermocouple wire.

Referring again to FIG. 9A, a joint member 968 can attach the treatment device 100 to the shaft 952. In one embodiment, the joint member 968 is crimped at both ends to fixedly attach the tubular electrode 102 to the shaft 952. In other embodiments, adhesives, fasteners, or other suitable features can permanently or semi-permanently attach the tubular electrode 102 to the shaft 952. The joint member 968 can be formed from an insulative material to electrically isolate the tubular electrode 102 from the shaft 952. In the illustrated embodiment, an insulative spacer element 969 mechanically (and electrically) separates the tubular electrode 102 from the shaft 952. In some embodiments, the joint member 968 and the spacer element 969 are an integrated part (e.g., a single molded part).

In operation, the actuator 953 can pull or release the control member 955 to, respectively, expand or contract the tubular electrode 102. For example, to expand the tubular electrode 102, the actuator 953 can pull the control member 955 toward the actuator 953 to urge the distal tip 956 towards the proximal end 963 of the tubular electrode 102. As the distal tip 956 is pulled in +X-axis direction, the tubular electrode 102 can correspondingly move radially outward in the +Y- and/or +Z-axis directions. To contract the tubular electrode 102, the actuator 953 can release the control member 955 to remove the tensile force at the distal tip 956. As the distal tip 956 moves in -X-axis direction, the tubular electrode 102 can correspondingly move radially inward in the −Y- and/or −Z-axis directions.

As discussed above, the slots 115 can be configured to bias the expansion and contraction of the tubular electrode 102. In particular, the slots 115 can expand the portion of the tubular electrode 102 in the electrode region 116a (FIG. 1) into a helical/spiral shape, while the slots 115 of the transition region 116b (FIG. 1) drive the electrode region 116a outwardly during expansion. In some embodiments, the flexible region 116c (FIG. 1) does not substantially bias expansion, but increases flexibility of the tubular electrode 102 to facilitate placement of the tubular electrode within an anatomical vessel. In general, various aspects of biased expansion, including helically shaped expansion, are described further in PCT Publication No. WO/2012/061159, which is incorporated herein in its entirety by reference.

The field generator 962 can provide various forms of output energy to the contact surface 110, including continuous energy or pulsed energy. In one embodiment, the field generator 962 delivers electrical energy over a conductive path that includes an arterial wall (e.g., the arterial wall 838 of FIGS. 8A and 8B) and a ground pad placed externally on the patient for monopolar delivery of energy. In another embodiment, the field generator 962 delivers electrical energy over a conductive path that includes an arterial wall and an electrode that is generally co-located with the contact surface 110 for bipolar delivery of energy. Also, in one embodiment, the field generator 962 can be external to a patient. In another embodiment, however, the field generator 962 may be positioned internal to a patient. For example, a field generator 962 can include a battery-powered field generator that can be deployed internally within a patient.

In general, the output energy of the field generator can have shaped waveforms, such as AC waveforms, sinusoidal waves, cosine waves, combinations of sine and cosine waves, DC waveforms, DC-shifted AC waveforms, RF waveforms, microwaves, ultrasound, square waves, trapezoidal waves, exponentially-decaying waves, and combinations thereof. When outputting a pulsed electrical field, the field generator 962 can be configured to output pulse widths of any desired interval, such as up to about 1 second. Suitable pulse intervals include, for example, intervals less than about 10 seconds. In addition, the field generator 962 can deliver a range of field strengths up to, for example, 10,000 V/cm. A person of ordinary skill in the art will recognize that a variety of waveforms and energies can be delivered depending on the procedure.

### CONCLUSION

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown or described in detail to avoid unnecessarily obscuring the description of the embodiments of the present technology. Although steps of methods may be presented herein in a particular order, alternative embodiments may perform the steps in a different order. Similarly, certain aspects of the present technology disclosed in the context of particular embodiments can be combined or eliminated in other embodiments. While advantages associated with certain embodiments of the present technology may have been disclosed in the context of those embodiments, other embodiments can also exhibit such advantages, and not all embodiments need necessarily exhibit such advantages or other advantages disclosed herein to fall within the scope of the present technology. Accordingly, this disclosure and associated technology can encompass other embodiments not expressly shown or described herein.

Certain aspects of the present technology may take the form of computer-executable instructions, including routines executed by a controller or other data processor. In some embodiments, a controller or other data processor can be specifically programmed, configured, or constructed to perform one or more of these computer-executable instructions. Furthermore, some aspects of the present technology may take the form of data, e.g., non-transitory data, stored or distributed on computer-readable media, including magnetic or optically readable or removable computer discs as well as media distributed electronically over networks. Accordingly, data structures and transmissions of data particular to aspects of the present technology are encompassed within the scope of the present technology. The present technology also encompasses methods of both programming computer-readable media to perform particular steps and executing the steps.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout to mean including at least the recited feature(s) such that any greater number of the same feature and/or additional types of other features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

I/we claim:

1. A catheter, comprising:
a tubular electrode having a wall, a contact surface defined by the wall, and cut shapes at least partially extending through the wall, wherein—
the tubular electrode is configured to transmit electrical energy to a treatment site within a body lumen via the contact surface,
the contact surface has a periphery and an interior region within the periphery, and
the individual cut shapes are configured to draw a portion of the electrical energy toward the interior region; and
a shaft having a distal end portion operably coupled to the tubular electrode, wherein the shaft is configured to locate the tubular electrode at the treatment site.

2. The catheter of claim 1 wherein:
the tubular electrode is expandable from a contracted state to an expanded state; and
when the tubular electrode is at the treatment site and in the expanded state, the edge portions are at least proximate to an inner surface of the body lumen.

3. The catheter of claim 1 wherein:
the tubular electrode includes slots extending at least partially through the wall of the tubular electrode;
the individual slots terminate in the individual cut shapes; and
the slots are configured to bias expansion of the tubular electrode toward the expanded state.

4. The catheter of claim 1 wherein the cut shapes are at least partially curved.

5. The catheter of claim 1, further comprising a spacer member operably disposed between the tubular electrode and the shaft, wherein the spacer member is configured to electrically isolate the tubular electrode from the shaft.

6. The catheter of claim 1, further comprising a control member operably coupled to the tubular electrode, and wherein:
the control member is configured to expand the tubular electrode toward the expanded state; and
the control member is further configured to supply electrical energy to the tubular electrode.

7. A catheter, comprising:
an elongated shaft having a distal end portion; and
a tubular electrode having a proximal end portion operably connected to the shaft via the distal end portion of the shaft, wherein the tubular electrode includes—
a wall having a contact surface, and
cut shapes at least partially extending through the wall, wherein the cut shapes include edge portions that extend towards an interior of the contact surface to at least partially define energy distribution regions,
wherein—
the electrode is transformable between a low-profile configuration for delivery to a treatment site within a body lumen of a human patient and a deployed configuration for treatment of the patient, and
when the electrode is at the treatment site and in the deployed configuration, the edge portions of the energy distribution regions are configured to deliver electrical toward an inner surface of the body lumen.

8. The catheter of claim 7 wherein the cut shapes are configured to define a continuous lesion profile at least at a portion of the inner surface of the body lumen.

9. The catheter of claim 7 wherein the cut shapes are configured to define a segmented lesion profile at least at a portion of the inner surface of the body lumen.

10. The catheter of claim 7 wherein the edge portions include curved segments that are configured to a least partially define the energy distribution regions.

11. The catheter of claim 7 wherein the cut shapes are differently shaped to at least partially define the energy distribution regions.

12. The catheter of claim 7 wherein the cut shapes are differently sized to at least partially define the energy distribution regions.

13. The catheter of claim 7 wherein:
the tubular electrode has a longitudinal axis; and
the cut shapes are spaced along the longitudinal axis to at least partially define the energy distribution regions.

14. The catheter of claim 7 wherein:
the tubular electrode includes a dielectric material covering an outer surface of the wall; and
at least a portion of the dielectric material is configured to impede a flow of electrical current between the tubular electrode and at least a portion of the inner surface of the body lumen.

15. A treatment device, comprising:
a tubular electrode have a wall with an outer surface and a contact surface located at the outer surface, wherein the tubular electrode is configured to receive electrical energy and to provide at least a portion of the electrical energy at the contact surface for delivering an electrical field toward a treatment site at an inner surface of a body lumen of a human patient;
at least one outer dielectric material portion at least partially covering the outer surface; and
at least one inner dielectric material portion at least partially covering the outer surface and configured to impede a flow of electrical current between the contact surface and the inner surface of the body lumen.

16. The treatment device of claim 15 wherein the outer and inner dielectric material portions are configured to impede a flow of current towards at least a portion of the inner surface of the body lumen.

17. The treatment device of claim 15 wherein:
the tubular electrode is expandable from a contracted state to an expanded state; and
when the tubular electrode is at the treatment site and in the expanded state, the contact surface is at least proximate to the inner surface of the body lumen.

18. The treatment device of claim 15 wherein:
the tubular electrode includes slots extending at least partially through the wall of the tubular electrode; and
the slots are configured to bias expansion of the tubular electrode toward the expanded state.

19. The treatment device of claim 15 wherein:
the individual slots terminate in individual cut shapes; and
the individual cut shapes are configured to draw a portion of the electrical energy toward an interior of the contract surface.

20. The treatment device of claim 15 wherein the individual cut shapes include edge portions that extend towards the interior of the contact surface.
